(19) United States
(12) Patent Application Publication
(10) Pub. No.: US 2006/0143766 A1
(43) Pub. Date: Jul. 6, 2006

(54) Ergonomic, temple-vented, lightweight, anti-glare eye shield & method of manufacture thereof

(76) Inventor: Joseph Danton Ramsey, Santa Monica, CA (US)

Correspondence Address: LAW OFFICES OF CLEMENT CHENG, 17220 NEWHOPE STREET #127, FOUNTAIN VALLEY, CA, 92708, US

(21) Appl. No.: 11/285,536

(22) Filed: Nov. 22, 2005

Related U.S. Application Data
(60) Provisional application No. 60/630,491, filed on Nov. 23, 2004.

(51) Int. Cl. A61F 9/00

(52) U.S. Cl. 2/15

(57) ABSTRACT

An ergonomic, temple-vented, lightweight, anti-glare eye protective device. The eye protection device is intended for use during procedures in the dental, medical and related professions to protect patients' eyes from splash, splatter, particles, bacteria and/or foreign matter. The shield may include a one-piece, flat, very thin, lightweight, highly flexible, flared-oblong shaped, transparent, tinted thermoplastic material with strategically placed foam “bumper pads” and two independent, elastic ear loops that serve as the means of attachment. The left and right sides of the shield are fully open, i.e., they do not come in contact with the wearer's face, affording total lateral ventilation in the area of the temples. The device can be adapted to be worn over the patient's eyeglasses if desired. Due to the low-cost manufacturing method, the device can he used economically on a disposable basis.

This application claims priority from U.S. provisional application Ser. No. 60/630,491 filed on Nov. 23, 2004.

### COPYRIGHT AUTHORIZATION

A portion of the disclosure of this patent document contains material that is subject to copyright and/or mask work protection. The copyright and/or mask work owner has no objection to the facsimile reproduction by anyone of the patent document or the patent disclosure, as it appears in the Patent and Trademark Office patent file or records, but otherwise reserves all copyright and/or mask work rights whatsoever.

### BACKGROUND OF THE INVENTION

1. Field of the Invention

This invention relates to eyewear and more particularly to an eye shield that provides mechanical and ventilated eye protection in a form that is readily and generally inexpensively produced.

2. Discussion of Related Art

Eyeglasses and goggles provide means by which eyes may be protected from flying debris, splashing liquid and the like. Such eyewear is generally burdensome or obstructive in certain circumstances, such as for patient eye protection during dental or medical procedures. While such eyewear may be useful on the factory floor or in the workshop, the obtrusive nature of such eyewear may be an impediment to its use in close quarters or where less substantial protection is needed.

The human eye is very sensitive and even the slightest or smallest amount of debris can cause discomfort or reaction. For the former, discomfort can arise from even delicate particles of dust while for the latter; grains of tiny pollen can evoke a strong allergic reaction. Where there is the risk of foreign material being introduced into the eye, it makes sense to provide protection for it, especially where the eyebrows, eyelashes, and eyelids are insufficient to provide such protection.

In minor medical procedures where the patient is awake or otherwise, eye protection may be needed. One example is during dental procedures where flying debris may occur as by a dentist's drill dislodging filling material. Despite the best care of the dentist, debris may travel beyond the patient's mouth, into the air, and possibly land in, on, or near the eye. This can be disconcerting to dental patients who might initially be hesitant to visit a dentist due to discomfort, Such inadvertent, although possibly minor, additional discomfort may lead patients to avoid the dentist or dental procedures.

Consequently, it may be very helpful to provide such patients and others eye shields or other eye protection to prevent the introduction of foreign material into the eye. Attempts have been made in the past to achieve such eye protection, but room for improvement still exists.

Some such prior attempts are set forth below and include the following publications and/or references:

| Patent/Publication | | Date of Issue/ |
|---|---|---|
| Number | Inventor | Publication |
| 836,796 | Anderson | 11/27/1906 |
| 1,123,375 | Rextrew | 01/05/1915 |
| 1,163,290 | Weeks | 12/07/1915 |
| 1,188,679 | Rextrew | 06/27/1916 |
| 1,203,079 | Van Antwerp | 10/31/1916 |
| 4,610,036 | LaPrairie | 09/09/1986 |
| Des. 319,451 | Russell | 08/27/1991 |
| 5,140,710 | Rademacher | 08/25/1992 |
| 5,245,709 | Shipcott | 09/21/1993 |
| 5,302,977 | Markovitz et al. | 04/12/1994 |
| 6,019,103 | Carroll | 02/01/2000 |
| 6,532,598 | Cardarelli | 03/18/2003 |

A more in-depth description of each of the more pertinent publications follows.

The Russell U.S. Design Pat. No. Des. 319,451 is directed to an eye shield that is flat, does not distribute force or pressure arising from attachment to the face over the surface of the eye shield (but instead focuses it horizontally across the center of the eye shield), and does not provide for distributed ventilation at its top and bottom. Instead, ventilation is obstructed at the top by an apparent pad. No means of attachment are indicated, but apertures for apparently connecting same are present on opposite sides of the eye shield,

The Markoyitz et al. U.S. Pat. No. 5,302,977 is directed to eyewear, such as sunglasses, which has a self-adjusting ear stem system. The eyewear has upper and lower frame members which contain an elastic cord means disposed therethrough which the elastic cord means can be extended on opposite sides of the wearer's face to fit around the wearer's ears.

In U.S. Pat. No. 5,140,710 issued to Rademacher, the reference is directed to an eye is shield having two layers of X-radiation protective material Is disclosed. X-radiation initially encounters a metalized thin layer that permits the substantial transmission of accompanying visible light yet partially diffracts, reflects, and refracts the X-radiation to decrease the amount of X-radiation passing through this metalized layer. Beneath the metalized thin layer is a lead layer that allows the passage of limited amounts of visible light yet functions to absorb effectively X-radiation. In combination, these two layers protect the eyes of a wearer from harmful X-radiation while permitting the wearer limited forward vision. The disposable layer in the eye shield may decrease the risk of propagating or transfening communicable diseases or agents.

In U.S. Pat. No. 1,188,679 to Rextrew, the reference is directed to an eye shield having ear loops used to attached the eye shield to the face.

In U.S. Pat. No. 5,245,709 to Shipcott, the reference is directed towards protective eyewear for a board user including a substantially thin, one-piece body made from a clear, flexible material having color and ultraviolet protection added thereto. The body includes wing shaped edges which fit over the temples of a wearer and have an elastic headband secured to is their ends for retaining the eyewear on the of the wearer. A plurality of resilient members are secured to the interior of the body so as to fit against the face of a user in the wearing position and to provide a plurality of spaces for the passage of air and water between the interior surface of the body and the face of the user. The headband is provided with a leash means having a strap secured thereto, for fixing of the eyewear to the neck of the wearer by means of a collar connected to the strap, or by means of the strap being directly connected to a garment worn by the user.

In U.S. Pat. No. 6,532,598 to Carthrelli, the reference is directed towards a lightweight and inexpensive mask providing protection to the nose and the eyes of dental patients from contaminants that are present in the dental office. The mask has a nose portion having an elasticized cord traversing across the philtrum area of a patient thereby causing the mask to adopt a triangular shape about the nose. The nose portion is formed to create natural ventilation along the patient nasolabial groove areas. The mask further includes a pair of transparent eye panels and means for affixing the mask on the patient's face.

In U.S. Pat. No. 6,019,103 to Carroll, the reference is directed to a disposable sanitary eye protector device that includes a molded, flexible plastic, face shield portion having two eye protector openings and nose receiving cut-out; two domed shaped clear plastic eye Protectors, one installed through each of the eye protector openings of the face shield portion; and an elastic securing cord having the cord ends thereof secured to the face shield portion to form a head encircling loop. The molded, flexible plastic, face shield portion is molded to the general contours of the eye and nose area of a wearer. Each of the two domed shaped, clear plastic eye protectors includes a transparent domed portion having an eye receiving cavity formed therein that is accessible through an eye insertion opening defined by an attachment ridge formed around the entire perimeter of the eye protector. In a preferred embodiment, a back surface of the face shield portion is coated with a restickable adhesive layer and a peel off cover to ensure the back surface of the face shield portion slightly adheres to the face of the wearer during user. The restickable adhesive layer may be impregnated with an antiseptic agent to kill contagions, such as bacteria and viruses that may accidentally contact the wearer's face.

### SUMMARY OF THE INVENTION

The present invention provides new eye shield technology that can be used to protect a patient's eyes during dental and other procedures.

The general purpose of the present invention, which will be described subsequently in greater detail, is to provide a new eye shield that is ergonomic, low-cost, vented, lightweight, and that reduces glares while protecting the eyes, in an eye shield which is not anticipated, rendered obvious, suggested, or even implied by any of the prior art eye shields, either alone or in any combination thereof.

The eye shield technology presented herein includes ergonomically designed, low-cost, temple-vented, lightweight, anti-glare eye protective devices to be used during procedures in the dental, medical and related professions to protect patients' eyes from bright light, splash, splatter, particles, bacteria and/or foreign matter. A method of making such eye shields is also disclosed.

The shield includes a one-piece, flat, very thin, lightweight, highly flexible, flared-oblong shaped, transparent, tinted thermoplastic material with strategically placed foam bumper pads” and two independent, elastic ear loops that serve as the means of attachment.

The left and right sides of the shield are fully open, i.e., they do not come in contact with the wearer's face, affording total lateral ventilation in the area of the temples. The device can be adapted to be worn over the patient's eyeglasses if desired. Due to the low-cost of the manufacturing method, the device can be used economically on a disposable basis.

The tinted anti-glare coating of the eye shield may serve to reduce brightness from overhead work and ceiling lights and to “soften” the experience of dental/medical procedures for the patient. As 100% of the total field of vision (forward and peripheral) is maintained, the wearer's view is not restricted in any way to avoid any possibility of claustrophobic response.

The eye shield attaches to the patient's bead by means of two separate, independent, closed “ear loops” on the right and left sides of the shield. Each ear loop is made of soft, highly elastic material that stretches around the back at the base of each ear and holds the eye shield in the proper position during procedures.

The ear loops replace the usual means of holding the eye shield in place, such as rigid temples or headbands extending out from the frame, and render the shield unobtrusive and unobstructive to dental or medical professionals during most, if not all, non-ocular procedures. Strategically located points of attachment for the two ear loops to the shield and the overall flared-oblong shape of the shield afford extended lateral protection of the patient's eyes, improved tension distribution across the shield and a stable, comfortable fit.

Working in conjunction with the above-described elements, “bumper pads” composed of low density, maximum compression plastic foam or similar material are strategically located along the top edge (brow) and bottom edge (nose/cheek) of the eye shield to provide maximum ventilation and comfort for the wearer. The ear loops may generally distribute tension advantageously across and among the “bumper pads,” or cushions.

The high degree of elasticity of the ear loop material in conjunction with the four strategically positioned ear loop anchors (indented approximately 2.0 cm from each side edge on top of shield and 1.0 cm from each side edge on bottom of shield) maximizes the range of lateral coverage required to acconlno˜te various head sizes. Due to the extended lateral range afforded by this design concept, two sizes of the eye shield are generally sufficient to provide adequate protection for the majority of wearers whose bead sizes fall within the normal child-to-large adult range.

The ear loops are attached to the shield's surface under the strategically “indented” (from the side edges) “bumper pads” on the upper (brow) edge and lower (nose/cheek) edge of the shield on the right and left sides of the shield.

The precise “indented” placement of the ear loop attachment produces (1.) Extended lateral protection for the eye region of the wearer's face, (2.) Wide-open lateral “temple air vents” on both sides of the shield and “vertical air vents” above and below the eyes which allow the device to be worn for long periods of time in comfort without any fogging or heat buildup, (3.) Maintenance of 100% of the wearer's peripheral vision.

### OBJECTS OF THE INVENTION

It is an object of the present invention to provide eye protection. It is another object of the present invention to provide eye protection in a convenient and comfortable manner. It is yet another object of the present invention to provide eye protection by means of an eye shield that is easily and inexpensively manufactured for rapid and ready use for medical procedures or otherwise. It is yet another object of the present invention to provide an eye shield that has an integrated shield portion as well as a number of cushions or bumpers, the eye shield attached to the face by means of elastic ear loops. These and other objects and advantages of the present invention will be apparent from a review of the following specification and accompanying drawings.

### BRIEF DESCRIPTION OF THE DRAWINGS

FIG. 1 is a right rear perspective view of an eye shield according to the present invention.

FIG. 2 is a full frontal elevational view of the eye shield in FIG. 1 showing the attachment or anchor areas of the ear loops to the shield.

FIG. 3 is a diagram of an alternative embodiment.

### DETAILED DESCRIPTION OF THE PREFERRED EMBODIMENTS

The detailed description set forth below in connection with the appended drawings is intended as a description of presently preferred embodiments of the invention and is not intended to represent the only forms in which the present invention may be constructed and/or utilized. The description sets forth the functions and the sequence of steps for constructing and operating the invention in connection with the illustrated embodiments. However, it is to be understood that the same or equivalent functions and sequences may be accomplished by different embodiments that are also intended to be encompassed within the spirit and scope of the invention.

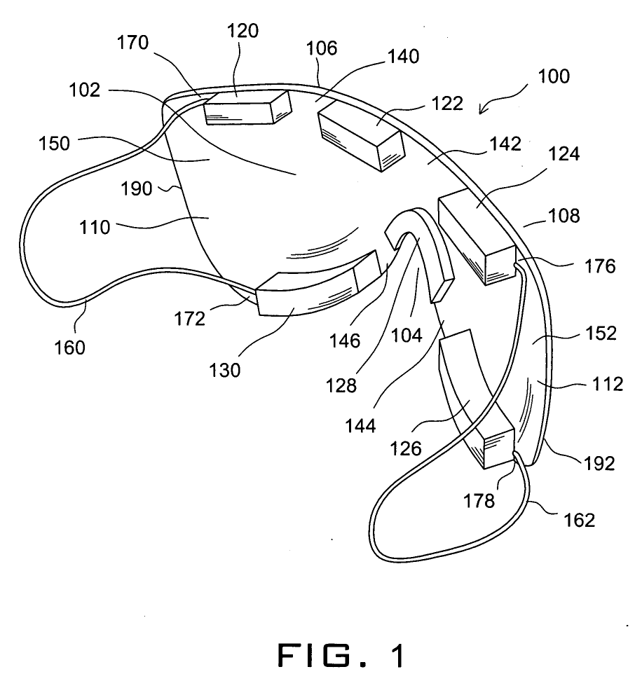

As shown in FIG. 1, the eye shield 100 set forth herein has a shield 102 that in a preferred embodiment may be tinted and include antiglare properties. The shield 102 is generally oblong in nature and has an indentation for the nose 104 between two eye covering portions; the left eye covering portion 106 and the right eye covering portion 108. Both the left and right eye covering portions 106, 108 generally include extended lateral protection to the outer sides of the shield 102. On the left side, an extended lateral protecting portion 110 is present; on the right side, a right extending lateral protecting portion 112 is present.

The shield 102 of the eye shield 100 has several cushions or bumpers including a left brow bumper pad 120, a center brow bumper pad 122, a right brow bumper pad 124, a right cheek bumper pad 126, a nose bridge pad 128, and a left cheek bumper pad 130.

As set forth in more detail below, the bumper pads, or cushions, are made of specially chosen materials in order to provide low contact pressure while also providing sufficient spacing and comfort as well as allowing ventilation about the shield 102. As shown in FIG. 1, vent slots are provided between the bumper pads or cushions, including: a left brow air vent 140 between the left brow bumper pad 120 and the center brow bumper pad 122: a right brow air vent 142 between the center brow bumper pad 122 and the right brow bumper pad 124; a right cheek air vent 144 between the right cheek bumper pad 126 and the nose bridge pad 128; and a left cheek air vent 146 between the nose bridge pad 128 and the left cheek bumper pad 130. Fully open temple air vents are provided on the left side 150 and the right side 152 between the left brow bumper pad 120 and the left cheek bumper pad 130 and between the right brow bumper pad 124 and the right cheek bumper pad 126, respectively.

Coupled to the shield 102, and as shown in FIG. 1 entrapped between corresponding left and right side brow and cheek bumper pads, are a left elastic eye loop 160 and a right elastic eye loop 162. The left and right elastic ear loops 160, 162 enable attachment of the shield 102 to a person's face when the person's nose is placed into the nose indentation 104 and the left and right elastic ear loops 160, 162 are fitted over and around the corresponding ear of the wearer. When the eye shield 100 is so fitted over the wearer's face, the bumper pads, or cushions, are slightly compressed and hold the shield 102 in a spaced-apart relation from the person's face but in closed proximity thereto, The extended lateral protecting flares 110, 112 serve to protect the lateral portions of the person's eyes while the main portion of the shield 102 protects the eyes towards their front.

The eye shield 100 incorporates in an integral and synergistic manner the elements set forth herein in order to provide an eye shield that is easily worn for extended periods as well as being easily manufactured.

The ergonomic, temple-vented, lightweight, anti-glare eye protective device 100 may be used to good advantage during procedures in the dental, medical and related professions to protect patients' eyes from splash, splatter, particles, bacteria and/or foreign matter. The shield 102 may have a one-piece, flat, very thin, lightweight, highly flexible, flared-oblong shaped, transparent, tinted thermoplastic material with strategically placed foam “bumper pads”, or cushions, and two independent, elastic ear loops that serve as the means of attachment, The left and right sides of the shield are fully open, i.e., they do not come in contact with the wearer's face, affording total lateral ventilation in the area of the temples. The device can be adapted to be worn over the patient's eyeglasses if desired. Due to the low cost manufacturing method, the device can be used economically on a disposable basis, but is durable enough to be used several times. Additionally, the eye shield is comfortable enough to be worn for extended periods with little or no discomfort.

The eye shield 100 may have a tinted anti-glare coating to reduce brightness from overhead work and ceiling lights and to “soften” the experience of dental or medical procedures for the patient. As 100% of the total field of vision (forward and peripheral) is maintained, the wearer's view is not restricted in any way to avoid any possibility of claustrophobic response.

The eye shield 100 attaches to the patient's head by means of two separate, independent, closed “ear loops” 160, 162 on the right and left sides of the shield. Each ear loop 160, 162 is made of soft, highly elastic material that stretches around the back at the base 20 of each ear to operate as an attacher thereto and holds the eye shield 100 in the proper position during procedures,

The ear loops 160, 162 replace the usual means of holding the eye shield 100 in place, such as rigid temples or headbands extending out from the frame, and render the shield generally unobtrusive and unobstructive to dental or medical professionals during all procedures. Strategically located points of attachment for the two ear loops 160, 162 and the overall flared-oblong shape of the shield 102 afford extended lateral protection of the patient's eyes, improved tension distribution across the shield and a stable, comfortable fit. Due to the mechanical and/or other interaction between the several components of the eye shield, a highly synergistic design for the eye shield may be achieved.

Working in conjunction with the above-described elements, “bumper pads” 120, 122, 124, 126, 128, 130 composed of low density, maximum compression plastic foam or similar material are strategically located along the top edge (brow) and bottom edge (nose/cheek) of the eye shield to provide maximum ventilation and comfort for the wearer. The pads also obstruct flying debris while allowing ventilation there between.

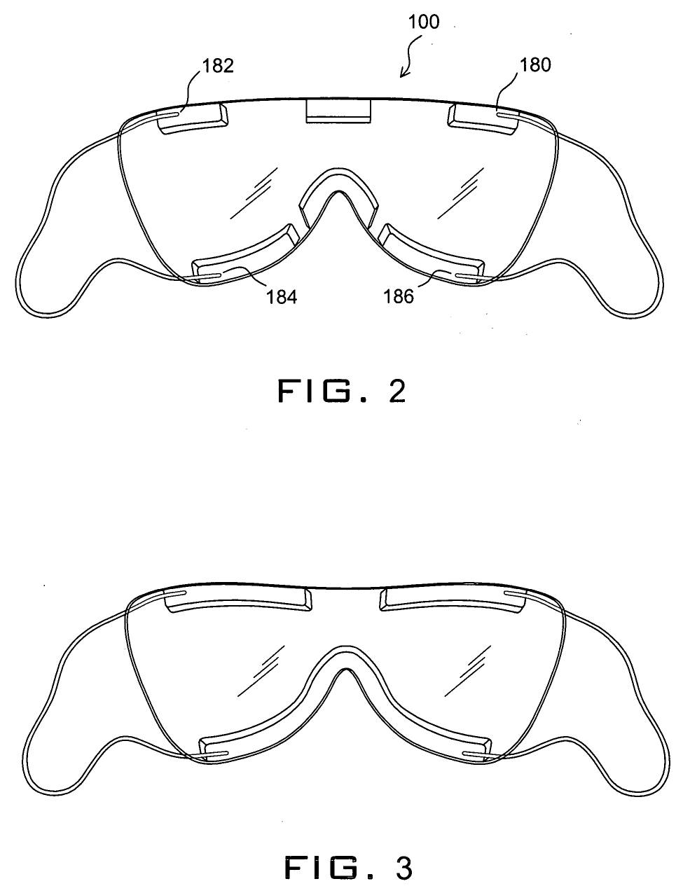

The high degree of elasticity of the ear loop material in conjunction with the four strategically positioned ear loop anchors 180, 182, 184, 186 (FIG. 2, indented approximately 2.0 cm from each side edge on top of shield and 1.0 cm from each side edge on bottom of shield) maximizes the range of lateral coverage required to accommodate various head sizes. Due to the significant lateral range afforded by this design concept, two sizes of the eye shield are sufficient to provide adequate protection for the majority of wearers whose head sizes fall within the normal child-to-large adult range.

The ear loops 160, 162 may be attached to the shield's surface under the strategically “indented” (from the side edges) “bumper pads” on the upper (brow) edge and lower (nose/cheek) edge of the shield on the right and left sides of the shield, The ear loops 160, 162 serve to position the shield 102 about the wearer's face, following Its curvature.

The precise “indented” placement of the ear loop attachment may produce (1.) Extended lateral protection for the eye region of the wearer's face, (2.) Wide-open lateral “temple air vents” on both sides of the shield and “vertical air vents” above and below the eyes which allow the device to be worn for long periods of time in comfort without any fogging or heat buildup, and (3,) Maintenance of 100% of the wearer's peripheral vision.

In order to achieve optimal performance in terms of adequate protection and wearer comfort as described above, the following ergonomic design elements of the eye shield may advantageously function as a system in a distinctly synergistic manner: Very thin (0.01-0.015 mm) tinted plastic shield 102 that bends easily to fit the contours of the wearer's face; and strategically placed, soft (high compression) foam bumper pads 120, 122, 124, 126, 128, 130 that allow for a well-ventilated, comfortable fit; the only points of contact with the wearer's face generally being above the eyebrows, on the frontal portion of the cheek bones and midway on the nose bridge. Variations in the size, shape, and thickness of the pads may allow the shield 102 to conform better or with more sensitivity to facial contours.

The two highly elastic ear loops 160, 162 increase the lateral size range of the eye shield 100 and attenuate the pressure of the loops on the back of the ears; and indentations 170, 172, 176, 178 from the shield edges 190, 192 to the side bumper pads/anchors on both the right and left side where the ear loops attach to the shield reduces further the ear loops' “pull” on the wearer's ears while allowing for extended lateral protection that extends beyond the eye sockets.

The precise spatial relationship between the bumper pads along the top (brow) section and the bottom (cheek) section of the shield and the indentation of the side ear loop anchor/pads create, respectively, (1.) vertical air vent spaces 140, 142, 144, 1445, and (2.) wide open lateral temple air vents 150, 152 at both sides 190, 192 of the shield. The resulting excellent ventilation for the wearer eliminates any heat buildup and/or fogging of the shield.

All of the above integrated design features of the invention combine to produce a low-cost, disposable, sensitive-fitting, eye protective device 100 that can be worn safely and comfortably by the patient and one which is completely unobtrusive to the dental or medical professional during treatment.

Critical features of an eye shield device as disclosed herein follow from fully integrated and interacting ergonomic design elements which include:

(1.) Proper fit to ensure full protection of the eye area.

(2.) Affording a high level of comfort (no heat buildup, no fogging, extremely low-pressure points of contact with the wearer's face) throughout even the longest dental/medical procedures.

(3.) Providing a means of patient eye protection that is completely unobtrusive and unobstructive to the dental/medical professional during care-giving procedures,

(4.) Low-cost and disposability

A heavier duty, reusable version of the invention would be suitable to provide eye protection in other applications and settings.

The above-described eye shield can be economically mass-produced in a relatively simple, low-cost, two-step manufacturing process;

1.) One-piece of appropriate thermoplastic material is stamped out in the exact size and contours of the shield 102.

2.) Bumper pads 120, 122, 124, 126, 128, 130 and ear loops 160, 162 are simultaneously attached to the shield using foam injection or other suitable means, the two ear loops 160, 162 being anchored by the four bumper pads 120, 130, 124, 126 located on the left 190 and right 192 sides at the top and bottom of the shield 102.

FIG. 1, 2 show a six pad configuration with three upper pads above three lower pads constituting the main embodiment. An alternate embodiment is seen in FIG. 3 that shows a three pad version of the full frontal view of the shield in flattened configuration. FIG. 3 shows the pad as seen from the perspective of a viewer opposite the user. As seen in FIG. 3 a continuous bottom pad is attached to the shield. The bottom pad is of approximately 13 cm in total length as measured by the straight distance between the two endpoints. The bottom pad forms a cheek and nose bridge pad. The top two pads are brow pads extending approximately 6 cm and having between them a single brow vent.

In the alternative embodiment, the ear loop is approximately 23 cm total length and elastic as previously mentioned in the main embodiment. The alternate embodiment varies only in the manner specifically stated and is otherwise the same as the main embodiment in all other respects. While the present invention has been described with regards to particular embodiments, it is recognized that additional variations of the present invention may be devised without departing from the inventive concept.

1. A protective eye device for use by patients in the dental, medical, or related professions or for general personal use or otherwise, comprising:
a. a flat, flexible, lightweight, flared-oblong shape, tinted plastic eye shield that covers at least the total width of the wearer's face from above the eye brows down to and including the mid-to-lower cheek area;
b. two separate, closed ear loops on the right and left sides of the eye shield said ear loops attaching the shield to the patients head and holding it in position with minimal tension; and
c. a bumper pad system composed of variously shaped low density, maximum compression plastic foam or other similarly performing material with respect to the properties of fit, flexibility, comfort and stability of the eye shield;
said bumper pads located along the top (brow) section and bottom (nose/cheek) section of the eye shield; their spacing and precise location produce wide-open lateral temple air vents the full height of the shield on both sides and four or more vertical air vents along the top and bottom edges of the shield.

2. The protective eye device of claim 1, wherein:
a. the plastic eye shield is maintained at an optimally functional distance from the wearer's face by the plastic foam (or similar material) Bumper Pad System on the inside perimeter at the top of the shield (brow area) and the bottom of the shield (nose/cheek area);
b. the sensitivity of the bumper pad system to facial contours maximizes ventilation of the eye shield, renders it non-fogging and provides a cushioning effect that increases the wearer's comfort.

3. The protective eye device of claim 1, wherein: each of the two ear loops is made of a soft, highly-elastic material that stretches around the back at the base of each ear and stabilizes the eye shield in the optimal position on the wearer's face;
each of the two ear loops is anchored strategically on the top and bottom at each side of the eye shield at a specific distance of indentation to enhance the flexibility, fit, arid coverage of the shield;
the specific points for the attachment of the ear loops are incorporated into the flared-oblong shape of the eye shield;
both ends of the two ear loops are securely attached to the eye shield by anchoring them between the four “indented” edge foam bumper pads (top and bottom) of the shield the plastic shield itself.

4. The protective eye device of claim 1, wherein the thinness of the plastic material of the eye shield permits it to gently conform to the facial contours of the eye area.

5. The protective eye device of claim 1, wherein the elasticity of the ear loops in conjunction with the shape and flexibility of the eye shield allow a given size to accommodate a wide range of facial widths.

6. A disposable protective eye device for use by patients in the dental, medical, or related professions or for general personal use, comprising:
a flat, flexible, lightweight, flared-oblong shape, tinted plastic eye shield having a flared oblong shape that covers at least the total width of the wearer's face from above the eye brows down to and including the mid-to-lower cheek area, the thinness of the plastic material of the eye shield permitting it to gently conform to the facial contours of the eye area which it covers;
first and second closed ear loops on the left and right sides of the eye shield, respectively, said ear loops attaching the shield to the patient's head and holding it in position with minimal tension;
each of the two ear loops made of a soft, highly-elastic material that 12 stretches around the back of each ear and stabilizes the eye shield in an optimal position on the wearer's face;
each of the two ear loops anchored strategically on the top and bottom at each side of the eye shield and at a specific distance of indentation to the respective edge of the eye shield to enhance the flexibility, tit, and coverage of the eye shield;
the specific points for the attachment of the ear loops incorporated into the flared-oblong shape of the eye shield;
the elasticity of the ear loops in conjunction with the shape and flexibility of the eye shield allow a given eye shield size to accommodate a wide range of facial widths;
a bumper pad system coupled to the eye shield and having variously shaped low density, maximum compression plastic foam or other material(s) which perform(s) similarly as bumper pads and with respect to the properties of fit, flexibility, comfort and stability of the eye shield;
both ends of the two ear loops securely attached to the eye shield by anchoring them between four indented edge foam bumper pads (top and bottom) of the shield the plastic shield itself;
the plastic eye shield maintained at an optimally functional distance from the wearer's face by the plastic foam (or similar material) bumper pad system on the inside perimeter at the top of the shield (brow area) and the bottom of the shield (nose/cheek area);
the sensitivity of the bumper pad system to facial contours maximizing ventilation of the eye shield, rendering it non-fogging and providing a cushioning effect that increases the wearer's comfort; and
the bumper pads intermittently located along the top (brow) section and bottom (nose/cheek) section of the eye shield, the spacing and location of the bumper pads producing wide-open lateral temple air vents the full height of the shield on both sides and four or wore vertical air vents along the top and bottom edges of the shield; whereby a protective eye shield is provided that flexibly conforms to a wearer's face according to gently tension placed upon it by the ear loops when worn.

7. An eye protector, comprising:
a flexible eye shield;
a cushion system intermittently spaced about a perimeter of said eye shield; and
first and second attachers coupled to left and right sides of said eye shield, respectively, for attaching said eye shield to a wearer; whereby attachment of said flexible eye shield to said wearer by said attachers flexes said eye shield and conforming it to a face of said wearer,

8. An eye protector as set forth in claim 7, wherein said flexible eye shield further comprises: a flat, flexible, lightweight, flared-oblong shape, tinted plastic eye shield having a flared oblong shape that covers at least the total width of the wearer's face from above the eye brows down to and Including the mid-to-lower cheek area, the thinness of the plastic material of the eye shield permitting it to gently conform to the facial contours of the eye area which it covers.

9. An eye protector as set forth in claim 5, wherein said first and second attachers further comprises:
first and second closed ear loops on the left and right sides of the eye shield, respectively, said ear loops attaching the shield to the patient's head and hold it in position with minimal tension;
each of the two ear loops made of a soft, highly-elastic material that stretches around the back of each ear and stabilizes the eye shield in an optimal position on the wearer's face;
each of the two ear loops anchored strategically on the top and bottom at each side of the eye shield and at a specific distance of indentation to the respective edge of the eye shield to enhance the flexibility, fit, and coverage of the eye shield;
the specific points for the attachment of the ear loops incorporated into the flared-oblong shape of the eye shield; and
the elasticity of the ear loops in conjunction with the shape and flexibility of the eye shield allow a given eye shield size to accommodate a wide range of facial widths.

10. An eye protector as set forth in claim 8 wherein said cushion system further comprises:
a bumper pad system coupled to the eye shield and having variously shaped low density, maximum compression plastic foam or other material performing similarly as bumper pads and with respect to the properties of fit, flexibility, comfort and stability of the eye shield;
the first and second attachers securely attached to the eye shield by anchoring them between four indented edge foam bumper pads (top and bottom) of the eye shield;
the eye shield maintained at an optimally functional distance from the wearerts face by the plastic foam (or similar material) bumper pad system on the inside perimeter at the top of the eye shield (brow area) and the bottom of the eye shield (nose/cheek area);
the sensitivity of the bumper pad system to facial contours maximizing ventilation of the eye shield, rendering it non-fogging and providing a cushioning effect that increases the wearer's comfort; and
the bumper pads intermittently located along the top (brow) section and bottom (nose/cheek) section of the eye shield, the spacing and location of the bumper pads producing wide-open lateral temple air vents the full height of the shield on both sides and four or more vertical air vents along the top and bottom edges of the shield.
